(19) United States
(12) Patent Application Publication
(10) Pub. No.: US 2007/0160635 A1
(43) Pub. Date: Jul. 12, 2007

(54) Cosmetic composition textured with a specific asymmetric bis-urea derivative

(75) Inventors: Sandrine Chodorowski-Kimmes, Senlis (FR); Nawel Baghdadli, Massy (FR); Mireille Arnaud-Roux, Aulnay (FR); Goulnara Garipova, La Chapelle Saint Mesmin (FR); Laurent Bouteiller, Bourg La Reine (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/633,034

(22) Filed: Dec. 4, 2006

Related U.S. Application Data
(60) Provisional application No. 60/761,335, filed on Jan. 24, 2006.

(30) Foreign Application Priority Data
Dec. 13, 2005 (FR) 0553849

(51) Int. Cl. A61K 8/44; A61K 8/42

(52) U.S. Cl. 424/401; 424/70.31

(57) ABSTRACT

The present invention relates to a cosmetic composition with a liquid fatty phase, comprising, in a physiologically acceptable medium, at least one liquid fatty phase, in which the said phase is textured with an effective amount of at least one asymmetric bis-urea compound of defined formula.

This non provisional application claims the benefit of French Application No. 05 53849 filed on Dec. 13, 2005 and U.S. Provisional Application No. 60/761,335 filed on Jan. 24, 2006.

The present invention relates to the cosmetic field and more particularly to a cosmetic composition with a liquid fatty phase textured with an asymmetric bis-urea compound.

To structure oils and give them the desired texture or viscosity, it is well known to those skilled in the art to use organogelling agents. Organogelling agents modify the molecular interactions in the oil and change its physical and/or chemical characteristics. However, the dissolution of these organogelling molecules in an oil or an oil mixture often requires a high temperature, which may give rise to additional heating costs and above all may be incompatible with the presence of heat-sensitive molecules. Furthermore, the gel obtained under these conditions does not always have the required stability over time.

It is known to those skilled in the art that compounds containing ureas and more particularly bis-ureas are good organogelling agents. Mention may be made, for example, of Feringa et al. in Chem. Eur. J, 1997, 3, 1238-1243, or Hanabusa et al. in Chem. Lett., 1996, 885-886 describing bis-ureas as gelling agents for organic solvents. Similarly, Andrew D. Hamilton describes in Tetrahedron Letters 39 (1998) 7447-7450 organogelling agents based on bis-urea molecules that are capable of gelling various organic solvents, including ethyl acetate.

The use of certain symmetric and asymmetric bis-ureas as organogelling agents has also been envisaged in documents WO 02/47628 and JP 2003-064346. These documents describe, inter alia, the use of symmetric and/or asymmetric bis-ureas for gelling cosmetic or non-cosmetic media. However, the symmetric and asymmetric bis-ureas described in these documents do not all dissolve at room temperature and/or in all cosmetic oils. To identify a gelling agent capable of satisfactorily gelling a particular cosmetic composition and capable of being dissolved at room temperature in the oils of the composition, many tests must be performed beforehand.

There is thus a need for a formula of organogelling molecules that may be termed universal in so far as it combines molecules of similar chemical structure, which prove to be effective, respectively, for gelling, at room temperature, at least one and advantageously several different cosmetic oils.

The object of the present invention is, precisely, to propose novel asymmetric bis-ureas that satisfy these requirements.

Consequently, according to one of its aspects, the invention relates to a cosmetic composition comprising, in a physiologically acceptable medium, at least one liquid fatty phase, characterized in that the said phase is textured with an effective amount of at least one compound of formula (I): 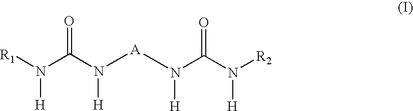
in which A, R₁ and R₂ are as defined below.

Unexpectedly, the inventors have discovered that the asymmetric bis-urea compounds under consideration according to the invention are gelling agents of choice for satisfying the need stated previously.

According to another of its aspects, the invention relates to the use of at least one asymmetric bis-urea compound as described above for structuring a cosmetic composition.

According to yet another of its aspects, the invention relates to a process for making up and/or caring for a keratin material, especially bodily or facial skin, the nails, the eyelashes and the hair, comprising the application to the surface to be treated of a cosmetic composition comprising at least one asymmetric bis-urea compound as described above.

Bis-Ureas

As stated above, bis-urea compounds more particularly considered according to the invention correspond to the general formula (I) below: 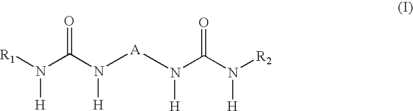
in which:
A is a group of formula: 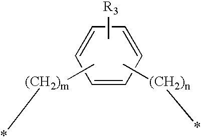
   with
R₃ being a hydrogen atom or a linear or branched C₁ to C₄ alkyl radical,
n and m being, independently of each other, equal to 0 or 1, and
* symbolizing the point of attachment of the group A to the two nitrogen atoms of the residue of the compound of general formula (I),
R₁ is a saturated or unsaturated, non-cyclic branched C₃ to C₁₅ carbon-based radical optionally containing from 1 to 3 heteroatoms chosen from 0, S, F and N and/or a carbonyl, and combinations thereof,
R₂ is different from R₁ and is chosen from linear, branched or cyclic, saturated or unsaturated C₁-C₂₄ alkyl radicals optionally containing from 1 to 3 heteroatoms chosen from O, S, F and N, and optionally substituted with:
1, 2 or 3 hydroxyl radicals,
an ester radical (—COOR₄), with R₄ being a linear or branched alkyl radical containing from 1 to 8, especially 1 to 6 or even 2 to 4 carbon atoms;
a saturated, unsaturated or aromatic cyclic radical containing from 5 to 12 carbon atoms, in particular a phenyl radical optionally substituted with one or more radicals, which may be identical or different, chosen from C₁-C₄ alkyl and trifluoromethyl radicals, or a morpholine derivative, and/or
one or more linear or branched C₁-C₄ alkyl radicals,
   or a salt or isomer thereof.

In particular, n and m are equal, and more particularly equal to zero, and R₃ is a radical R′₃ as defined below. Thus, preferably, A represents a group

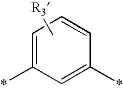

with R₃′ being a linear or branched C₁ to C₄ alkyl radical and * symbolizing the points of attachment of the group A to the two nitrogen atoms of the residue of the compound of general formula (I).

According to one variant of the invention, the compound of general formula (I) comprises, as A, at least one group chosen from: 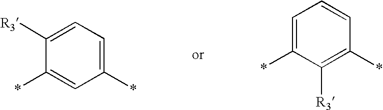
with R₃′ and * being as defined above.

In particular, R₃′ may be a methyl group, and in this case the group A represents a group

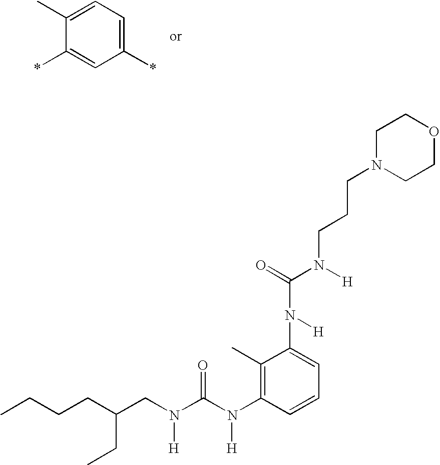

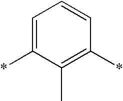

* being as defined above.

In particular, the compounds are such that A is a mixture of 2,4-tolylene and 2,6-tolylene, especially in (2,4 isomer)/(2,6 isomer) proportions ranging from 95/5 to 80/20.

According to one embodiment of the invention, the compound of general formula (I) comprises, as R₁, a branched C₆-C₁₅ radical.

According to one embodiment of the invention, the compound of general formula (I) comprises, as R₁, a group chosen from: 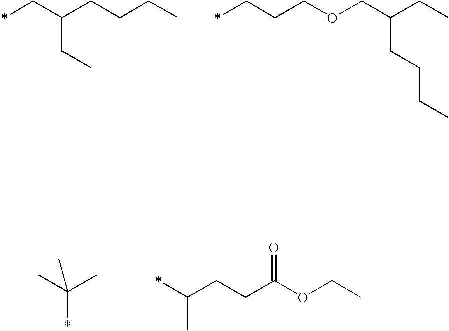
with * symbolizing the point of attachment of the group R₁ to the nitrogen of the residue of the compound of general formula (I).

As emerges from the examples below, the presence of one and/or other of the two radicals in the molecule of general formula (I) proves to be particularly advantageous for giving a universal nature within the meaning of the invention to the corresponding asymmetric bis-urea derivatives.

As regards R₂, which is different from R₁, it may be advantageously chosen from the following groups: 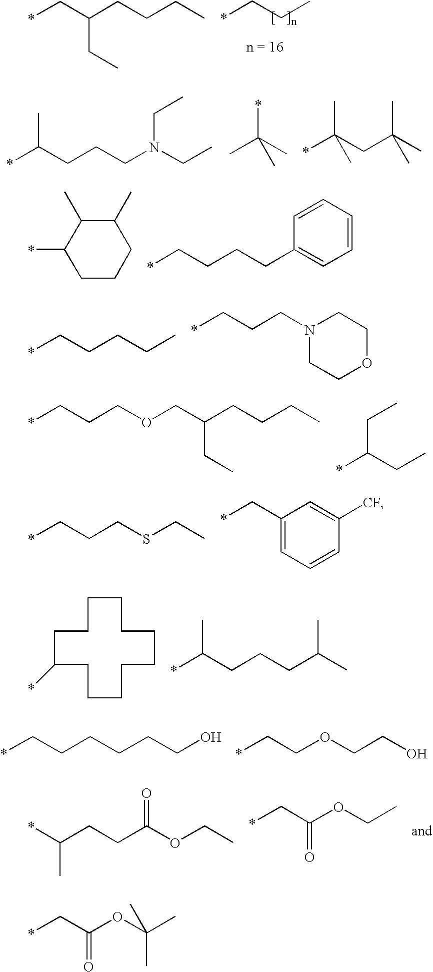
with * symbolizing the point of attachment of the group R₂ to the nitrogen of the residue of the compound of general formula (I).

As non-limiting illustrations of compounds that are most particularly suitable for the invention, mention may be made more particularly of the following compounds:

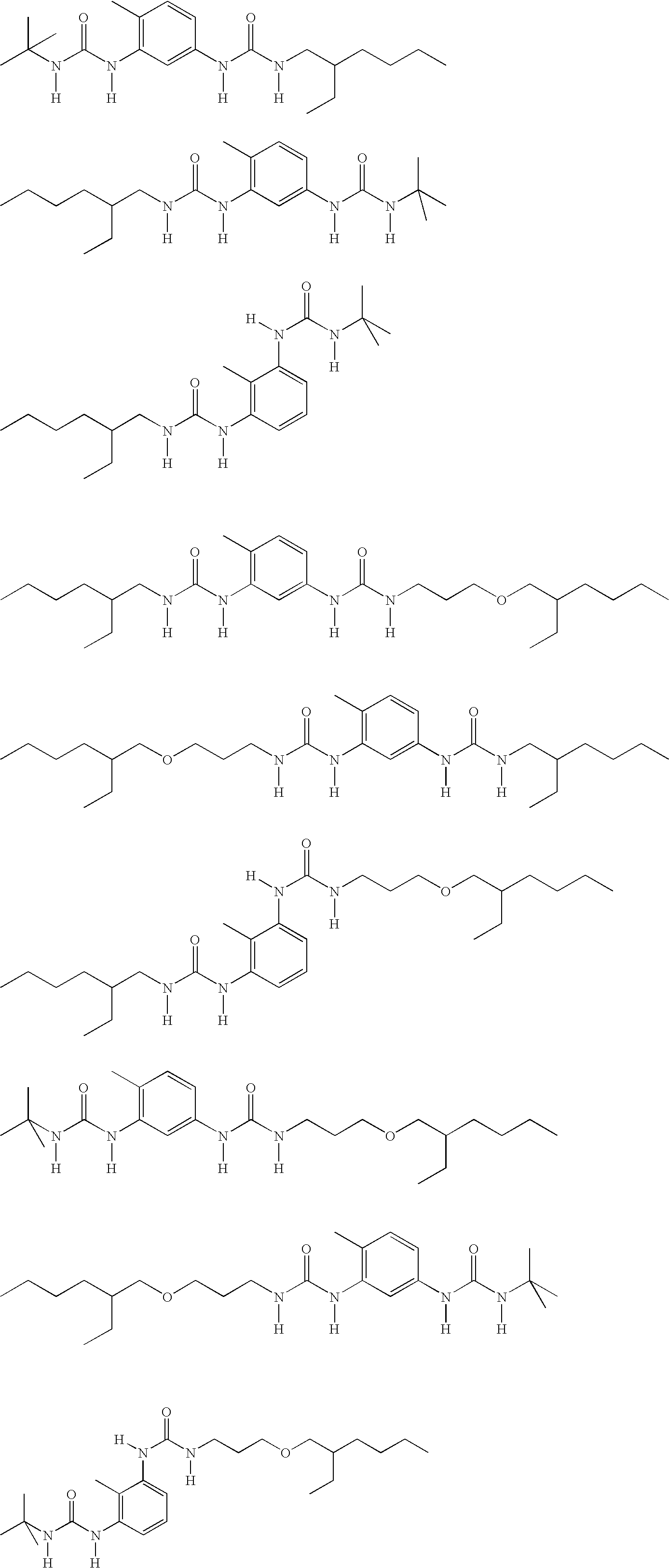

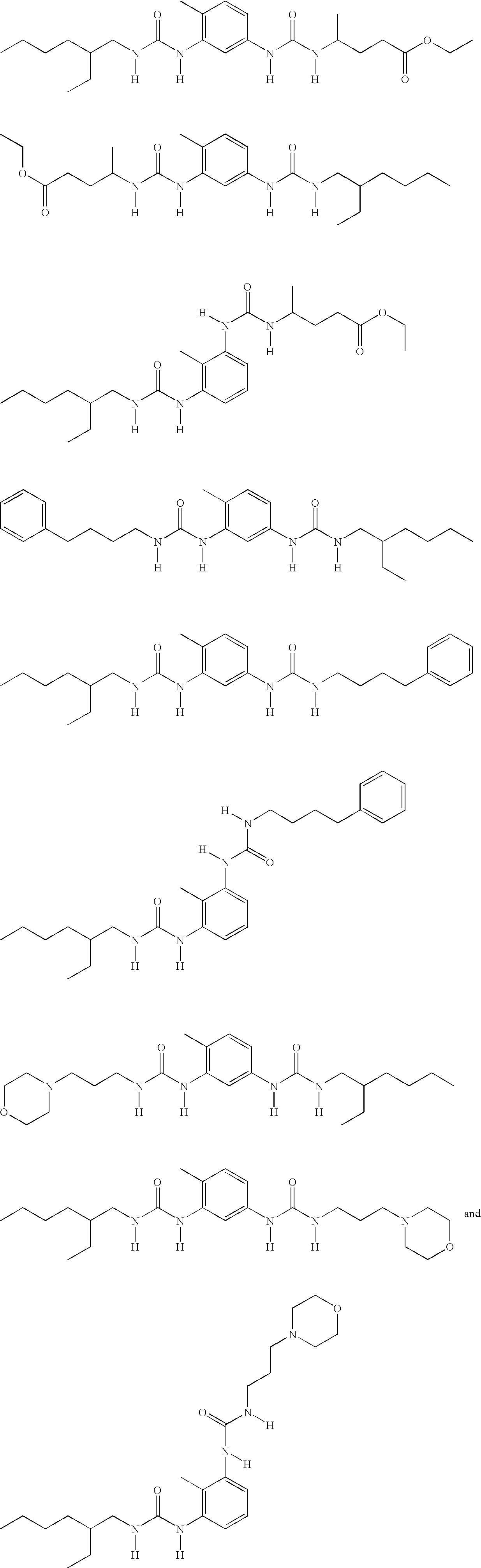

The expression “particularly suitable in the context of the invention” means that the compound of general formula (I), alone or as a mixture in various proportions, can dissolve at room temperature in several cosmetic oils and that it proves to be effective for gelling the said oil or oil mixture under consideration and thus giving it the desired physical and/or chemical properties.

For the purposes of the present invention, the term “effective amount” denotes the amount that is necessary and sufficient to obtain texturing of the oil or oil mixture under consideration in the composition according to the invention.

This texturing is reflected in particular by an increase in the viscosity, which may be due to the introduction of at least one compound of general formula (I).

For example, the compositions according to the invention may contain from 0.0001% to 5% by weight of asymmetric bis-urea, especially from 0.001% to 1% or even from 0.004% to 0.5% by weight of at least one asymmetric bis-urea relative to the total weight of the liquid fatty phase.

For obvious reasons, this effective amount is liable to vary significantly depending firstly on the nature of the substituents R₁ and/or R₂ of the bis-urea derivative, the positional isomer, and whether or not it is used in pure form or as a mixture with other bis-urea derivatives of formula (I), and secondly on the nature of the oily phase.

For reasons associated especially with the preparation procedure, the asymmetric bis-urea derivatives of general formula (I) may be used in the context of the present invention in the form of a mixture of derivatives of formula (I) with each other and/or with the two corresponding forms of symmetric bis-urea derivatives. For the purposes of the present invention, the term “symmetric bis-ureas” means the bis-ureas according to formula (1) with identical radicals R₁ and R₂.

In general, the compound of general formula (I) according to the invention is derived from the reaction between at least one diisocyanate of formula: 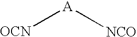
and at least two different primary amines of formulae: 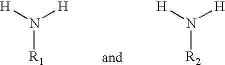
with A, R₁ and R₂ as defined above.

Where appropriate, the various diisocyanates may be positional isomers of the substituent R₃ on the group A, especially in 95/5 or 80/20 proportions.

The term “at least two different primary amines” means that other primary amines Rₓ—NH₂ in which Rx corresponds to the definitions proposed for R₁ and R₂, may be added thereto.

The number of amines used for the reaction may be greater than or equal to 2 and may range, for example, from 2 to 20 and more particularly between 3 and 10. To facilitate the preparation of the composition and the characterization of the mixture thus obtained, it is advantageous to remain limited to the use of two amines.

Preferably, the amines used are, as a whole, in a mole ratio of 2 to 3 equivalents and especially 2.1 to 2.5, or even 2.2 equivalents, per one equivalent of diisocyanate(s).

In the particular case where only two primary amines are used for the reaction, the mole ratio n(R₁)/n(R₂) may be between 1/99 and 99/1 and more particularly between 5/95 and 95/5, or even between 10/90 and 90/10, with n(R₁) corresponding to the number of moles of: 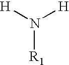
and n(R₂) corresponding to the number of moles of: 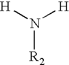
and with R₁ and R₂ as defined above.

The reaction is generally performed under an inert atmosphere, for example under argon, in an anhydrous medium with, for example, a reaction medium temperature maintained below 50° C., or even between 15° C. and 40° C. and preferably between 18° C. and 25° C.

The diisocyanate(s) may be dissolved in an anhydrous solvent such as tetrahydrofuran, 2-methyltetrahydrofuran, N-methylpyrrolidone, butyl acetate or methyl ethyl ketone to a concentration that may range from 1% to 30% by weight, preferably from 2% to 20%, or even from 4% to 10% by weight.

A solution containing the amines is generally prepared in the same solvent as the diisocyanate(s) to a concentration ranging, for example, from 0.1% to 99.9% by mass. The temperature of the reaction medium should preferentially not exceed 40° C. and the amine concentration and the rate of addition of the solution containing the amines should thus preferentially be adjusted to this need. The reaction medium may be left stirring, for example, for 30 minutes to 12 hours. The reaction progress may be monitored by infra red spectrometry (especially by observing the disappearance of the NCO band between 2250 and 2280 cm⁻¹). For example, at the end of the reaction, the reaction medium is poured into a large amount of acidic water (especially of pH 3-4 with HCl). A precipitate is then obtained, which is filtered off, washed, for example several times especially with water, and dried under reduced pressure, especially under vacuum or freeze-dried.

Consequently, after this reaction, at least one of the following two forms: 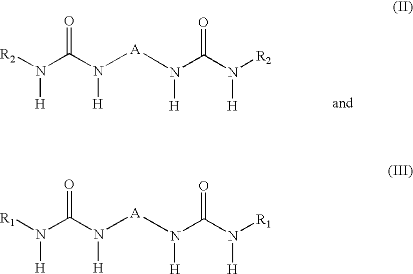
with A, R₁ and R₂ as defined above, with A being identical in formulae (I), (II) and (III), R₁ being identical in formulae (D) and (III) and R₂ being identical in formulae (I) and (II), is obtained together with the expected asymmetric derivative of formula (I).

The precipitate corresponds to the expected compounds of formula (I), and may be characterized by NMR spectrometry (¹H and/or ¹³C) and/or by HPLC and may be used in its native form for texturing the oily medium under consideration.

At the end of the reaction, the mixture of bis-ureas (I), (II) and (III) is isolated and may be used in its native form for gelling the desired oily medium.

In this case, the compound of formula (I) is used in the liquid fatty phase in the form of a mixture with the compounds of general formulae (ID) and (III).

The mixture of bis-ureas is advantageously soluble at a temperature of less than or equal to 50° C., or even less than or equal to 30° C., and especially at room temperature, in the liquid fatty phase to be textured.

Liquid Fatty Phase

The compositions according to the invention may comprise an oily phase comprising, for example, non-silicone oils and/or silicone oils.

For the purposes of the patent application, the term “liquid fatty phase” means a fatty phase that is liquid at room temperature (25° C.) and atmospheric pressure (760 mm Hg), composed of one or more mutually compatible fatty substances that are liquid at room temperature, also known as oils.

The oily phase may represent from 1% to 99.99% by weight of the composition, or even from 10% to 90%, and in particular from 20% to 50% by weight of the composition.

The liquid fatty phase may contain oils that may or may not be suitable for dissolving or for being gelled with a bis-urea compound as defined above. A person skilled in the art will take care to select the oils and the respective proportions thereof in the liquid fatty phase so as to obtain the desired dissolution of the bis-urea and gelation of the liquid fatty phase.

a. Silicone Oil

According to one variant of the invention, the liquid fatty phase comprises at least one silicone oil.

The silicone oils that may be used in the liquid fatty phase of the invention may also be linear non-volatile polydimethylsiloxanes (PDMSs), which are liquid at room temperature; polyalkylmethylsiloxanes and in particular polydimethylsiloxanes comprising alkyl, alkoxy or phenyl groups, which are pendent and/or at the end of a silicone chain, these groups each containing from 2 to 24 carbon atoms; dimethicone copolyols, alkylmethicone copolyols; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates, cetyl dimethicone, silicones containing alkylglyceryl ether groups, silicones containing amine side groups and dilauroyltrimethylolpropane siloxysilicate; fluorosilicones containing group(s) pendent or at the end of a chain, containing from 1 to 12 carbon atoms, all or some of the hydrogen atoms of which are replaced with fluorine atoms, such as silicones containing alkyl and perfluoroalkyl groups, silicones containing oxyethylene/oxypropylene (OE/OP) side groups and perfluoro groups, silicones containing perfluoro side groups and glycerolated side groups, and perfluoroalkylmethylphenylsiloxanes, and mixtures thereof.

Preferably, the silicone oil may be chosen from volatile cyclic silicones such as D4, D5 or D6, and phenyl trimethicones, and mixtures thereof.

The liquid fatty phase advantageously contains at least 5% and for example from 10% to 90% by weight of silicone oil(s).

b. Ester Oil

According to one variant of the invention, at least one of the oils of the liquid fatty phase is an “ester oil”, which is chosen from esters of monocarboxylic acids with monoalcohols and polyalcohols.

Advantageously, the said ester corresponds to formula (IV) below:
R₁—CO—O—R₂ (IV)
in which R₁ represents a linear or branched alkyl radical of 1 to 40 carbon atoms and preferably of 7 to 19 carbon atoms optionally comprising one or more ethylenic double bonds, and optionally substituted.

R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ may bear one or more substituents chosen, for example, from groups comprising one or more heteroatoms chosen from O, N and S, such as amino, amine, alkoxy or hydroxyl.

The number of carbon atoms in R₁+R₂ may be greater than or equal to 9.

Examples of groups R₁ are those derived from fatty acids, preferably higher fatty acids, chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters that may be used in the fatty phases of the compositions of the invention are, for example, purcellin oil (cetostearyl octanoate), isononyl isononanoate, isopropyl myristate, 2-ethylhexyl palmitate, 2-octyldodecyl stearate, 2-octyldodecyl erucate, isostearyl isostearate and heptanoates, octanoates, decanoates or ricinoleates of alcohols or of polyalcohols, for example of fatty alcohols.

Preferably, R₁ is an unsubstituted branched alkyl group of 4 to 14 carbon atoms and preferably of 8 to 10 carbon atoms and R₂ is an unsubstituted branched alkyl group of 5 to 15 carbon atoms and preferably of 9 to 11 carbon atoms. Mention may also be made of esters comprising 3 to 10 carbon atoms in total, such as ethyl acetate and/or butyl acetate.

Isopropyl N-lauroylsarosinate (Eldew 205-SL from Ajinomoto) may also be advantageous according to one aspect of the invention.

Advantageously, the liquid fatty phase comprises from 0.5% to 100% by weight, preferably from 1% to 80% by weight, more preferably from 2% to 50% by weight and better still from 3% to 40% by weight of ester oil(s).

C. Non-Silicone Oil

The liquid fatty phase of the compositions according to the invention may also contain one or more non-silicone oils different from the ester oils described above. These non-silicone oils may be chosen from the group of hydrocarbon-based oils and volatile ethers.

The non-silicone oil may also be chosen from fluoro oils such as perfluoropolyethers, perfluoroalkanes, for instance perfluorodecalin, perfluoro-adamantanes, perfluoroalkyl phosphate monoesters, diesters and triesters, and fluorinated ester oils.

The liquid fatty phase may also contain other non-silicone oils, for example polar oils such as:
hydrocarbon-based plant oils with a high triglyceride content consisting of fatty acid esters of glycerol in which the fatty acids may have varied chain lengths, these chains possibly being linear or branched, and saturated or unsaturated; these oils are especially wheatgerm oil, corn oil, sunflower oil, shea oil, castor oil, sweet almond oil, macadamia oil, apricot oil, soybean oil, rapeseed oil, cottonseed oil, alfalfa oil, poppy oil, pumpkin oil, sesameseed oil, marrow oil, avocado oil, hazelnut oil, grapeseed oil, blackcurrant pip oil, evening primrose oil, millet oil, barley oil, quinoa oil, olive oil, rye oil, safflower oil, candlenut oil, passionflower oil or musk rose oil; or alternatively caprylic/capric acid triglycerides, for instance those sold by the company Stearines Dubois or those sold under the names Miglyol 810, 812 and 818 by the company Dynamit Nobel,
C₆-C₄₀ synthetic ethers,
C₈-C₃₂ fatty alcohols, for instance oleyl alcohol or octyldodecanol,
C₈-C₃₂ fatty acids, for instance oleic acid, linoleic acid or linolenic acid, and
mixtures thereof.

The liquid fatty phase may also contain a polar oils such as linear or branched hydrocarbons or fluorocarbons, of synthetic or mineral origin, for instance liquid paraffins (such as C₈-C₁₆ isoparaffins, isododecane or isohexadecane) and derivatives thereof, petroleum jelly, polydecenes, and hydrogenated polyisobutenes such as parleam, squalane, and mixtures thereof.

Most particularly, the liquid fatty phase comprises at least one lipophilic compound chosen from the following list:
C₆-C₃₂, preferably C₈-C₂₈ and better still C₁₂-C₂₆ monoalcohols, preferentially octyldodecanol;
C₆-C₃₂, preferably C₈-C₂8 and better still C₁₂-C₂₆ branched alkanes, preferentially parleam of formula —CH₂—CH(CH₃)n- with n=4 to 8, and isododecane;
linear C₁₃-C₄₈, preferably C₁₈-C₄₀ and better still C₂₀-C₃₂ alkanes;
difunctional oils, comprising two functions chosen from ester and/or amide, containing 6 to 30 carbon atoms, especially 8 to 28 and better still 10 to 24 carbon atoms, and 4 heteroatoms chosen from O and N; preferably, the amide and ester functions are in the chain; and
mixtures thereof.

Generally, the non-silicone oils represent from 5% to 95% and better still from 20% to 75% of the total weight of the liquid fatty phase.

Needless to say, a mixture of silicone oils and carbon-based oils may be used, and in particular a mixture of at least one lipophilic compound chosen from the above list with a silicone oil (phenyl trimethicone, PDMS or volatile silicones).

Other Fatty Substances

The compositions according to the invention may also comprise at least one solid fatty substance, which may be chosen from waxes and pasty compounds.

For the purposes of the present invention, the term “wax” means a lipophilic compound that is solid at room temperature (25° C.), with a reversible solid/liquid change of state, having a melting point of greater than or equal to 30° C. and better still greater than 45° C., and which may be up to 120° C.

The waxes may be hydrocarbon-based waxes, fluoro waxes and/or silicone waxes and may be of plant, mineral, animal and/or synthetic origin.

As waxes that may be used in the first composition of the invention, mention may be made of beeswax, carnauba wax, candelilla wax, paraffin, microcrystalline waxes, ceresin or ozokerite; synthetic waxes, for instance polyethylene wax or Fischer Tropsch wax, and silicone waxes, for instance alkyl or alkoxydimethicones containing from 16 to 45 carbon atoms.

The compositions may also contain a micronized wax, also known as a micro wax.

As a guide, the compositions according to the invention may contain from 0.1% to 50% by weight and better still from 1% to 30% by weight of wax relative to their total weight.

More particularly, the compositions according to the invention may comprise from 0.1% to 40% by weight, especially from 0.1% to 30% by weight and more particularly from 0.5% to 25% by weight of solid fatty substance(s) relative to the total weight of the composition.

Aqueous Phase

The composition may, where appropriate, comprise at least one aqueous phase, which may or may not consist essentially of water.

The composition may also comprise a mixture of water and of water-miscible organic solvent (miscibility in water of greater than 50% by weight at 25° C.), for instance lower monoalcohols containing from 1 to 5 carbon atoms such as ethanol or isopropanol, glycols containing from 2 to 8 carbon atoms such as propylene glycol, ethylene glycol, 1,3-butylene glycol or dipropylene glycol, C₃-C₄ ketones and C₂-C₄ aldehydes.

The aqueous phase (water and possibly the water-miscible organic solvent) may be present in a content ranging from 1% to 95% by weight, especially ranging from 3% to 80% by weight and in particular ranging from 5% to 60% by weight relative to the total weight of the composition.

According to another embodiment in accordance with the invention, the preparation is free of water.

Dyestuffs

According to one embodiment, the composition according to the invention may also contain at least one organic or inorganic dyestuff, especially of pigment or nacre type.

According to another embodiment, the composition according to the invention may also contain at least one dyestuff chosen from lipophilic dyes, hydrophilic dyes, pigments, nacres and materials with a specific optical effect, and mixtures thereof.

The term “nacres” should be understood as meaning coloured particles of any form, which may or may not be iridescent, especially produced by certain molluscs in their shell or else synthesized, and which have a colour effect by optical interference.

This dyestuff may be present in a proportion of from 0.01% to 50% by weight relative to the total weight of the composition, in particular from 0.5% to 40%, more particularly from 5% to 25%, especially from 0.01% to 20%, in particular from 0.1% to 10% or even from 2% to 5% by weight relative to the total weight of the composition.

Other Additives

The composition of the invention may also comprise any ingredient usually used in the field under consideration.

Needless to say, a person skilled in the art will take care to select the optional additional ingredients and/or the amount thereof such that the advantageous properties of the composition according to the invention are not, or are not substantially, adversely affected by the envisaged addition.

Mention may be made especially of cosmetic or dermatological active agents, fillers, vitamins, thickeners other than those corresponding to the general formula (I), gelling agents other than those corresponding to the general formula (I), trace elements, softeners, sequestrants, fragrances, acidifying or basifying agents, preserving agents, sunscreens, surfactants, antioxidants, hair-loss counteractants, antidandruff agents, propellants and ceramides, or mixtures thereof.

The composition according to the invention may especially be in the form of a suspension, a dispersion, a solution, especially an organic solution, a gel, an emulsion, especially an oil-in-water (O/W) or water-in-oil (W/O) or multiple (W/O/W or polyol/O/W or O/W/O) emulsion or in the form of cream, paste, mousse, vesicular dispersion especially of ionic or nonionic lipids, two-phase or multiphase lotion, spray, powder, stick or wand.

A person skilled in the art may select the appropriate galenical form, and also the method for preparing it, on the basis of his general knowledge, taking into account firstly the nature of the constituents used, especially their solubility in the support, and secondly the intended use of the composition.

The composition according to the invention may be a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

The composition according to the invention may be a composition for protecting or caring for the skin of the face, the neck, the hands or the body, especially an anti-wrinkle or anti-fatigue composition, for making the skin radiant, a moisturizing or treating composition; an antisun or artificial tanning composition.

The composition according to the invention may also be a hair product, especially for holding the hairstyle or for shaping the hair. The hair compositions are preferably shampoos, gels, hairsetting lotions, blow-drying lotions, or fixing and styling compositions such as lacquers or sprays. The lotions may be packaged in various forms, especially in vaporizers or pump-dispenser bottles or in aerosol containers in order to apply the composition in vaporized form or in the form of a mousse.

Needless to say, the composition of the invention should be cosmetically or dermatologically acceptable, i.e. it should contain a physiologically acceptable non-toxic medium that may be applied to human skin, integuments or lips. For the purposes of the invention, the term “cosmetically acceptable” means a composition of pleasant appearance, odour and feel.

The composition according to the invention may be manufactured via the known processes generally used in cosmetics or dermatology.

A subject of the invention is also a cosmetic treatment process, especially for making up, cleansing, sun-protecting, shaping, dyeing or caring for keratin materials, especially bodily or facial skin, the nails, the hair and/or the eyelashes, comprising the application to the surface to be treated of a cosmetic composition as defined above.

The examples given below are produced as non-limiting illustrations of the field of the invention.

Materials and Methods

Unless otherwise indicated, the compounds of general formula (I) under consideration in each of the examples below were prepared according to the same procedure as described below.

The specific amounts adopted for the reagents are specified in the examples below.

To a solution of toluene diisocyanate in anhydrous THF is added a solution of the amine mixture in anhydrous THF. The addition is performed under argon, at room temperature, the rate of addition being such that the temperature of the reaction medium never exceeds 50° C. and more preferentially 40° C. The disappearance of the isocyanates is monitored by IR spectrometry by means of the disappearance of the band between 2250 and 2280 cm⁻¹. Once the diisocyanate has completely reacted, the reaction mixture is poured into water acidified to pH 3 with hydrochloric acid. The precipitate obtained is filtered off, washed several times with water and finally dried under vacuum or freeze-dried. A white powder is obtained with a molar yield ranging from 80% to 99%, and is used in its native form after analysis (HPLC coupled to mass spectrometry).

The examples given below differ in the molar proportions of the starting diisocyanate isomers used, the chemical nature of the amine(s) used in the reaction mixture and/or the proportion of one amine relative to the other in the starting mixture.

### EXAMPLE 1

The reaction mixture contains 50 g (0.28 mol) of tolylene diisocyanate as a mixture of the 2,4 isomer and the 2,6 isomer in a 95/5 proportion and a mixture of amines consisting of 80 mol % of 2-ethylhexylamine (m=63.6 g and n=0.49 mol) and 20 mol % of tert-butylamine (m=9 g and n=0.12 mol). The composition of the white powder obtained is determined by HPLC coupled to mass spectrometry, and does indeed contain the following mixture of products in the expected proportions:

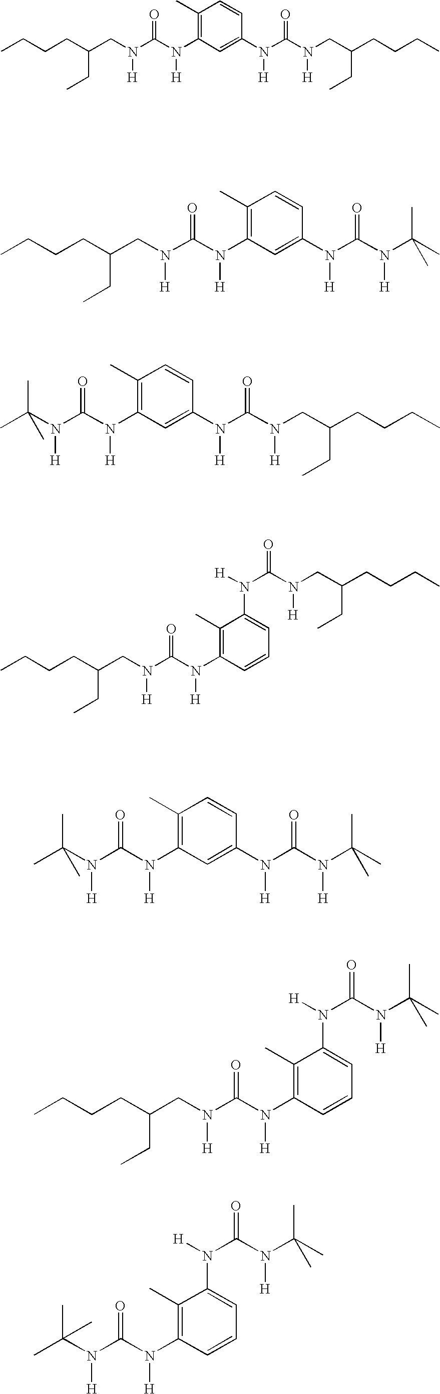

### EXAMPLE 2

The reaction mixture contains toluene duisocyanate consisting of a mixture of 2,4 and 2,6 isomers in a 80/20 proportion (m=50 g and n=0.28 mol) and a mixture of amines consisting of 80 mol % of 2-ethylhexylamine (m=63.6 g and n=0.49 mol) and 20 mol % of tert-butylamine (m=9 g and n=0.12 mol). The composition of the white powder obtained contains the same mixture of products as that obtained in Example 1 in different proportions (confirmation by HPLC coupled to mass spectrometry).

### EXAMPLE 3

The reaction mixture contains toluene diisocyanate as a mixture of the 2,4 isomer and the 2,6 isomer in a 95/5 proportion (m=15 g and n=86 mmol) and a mixture of amines consisting of 80 mol % of 2-ethylhexylamine (m=1.8 g and n=139 mmol) and 20 mol % of 3-(2-ethylhexyloxy)propylamine (m=6.5 g and n=35 mmol). The composition of the white powder obtained is determined by HPLC coupled to mass spectrometry, and contains the mixture of products below in the expected proportions:

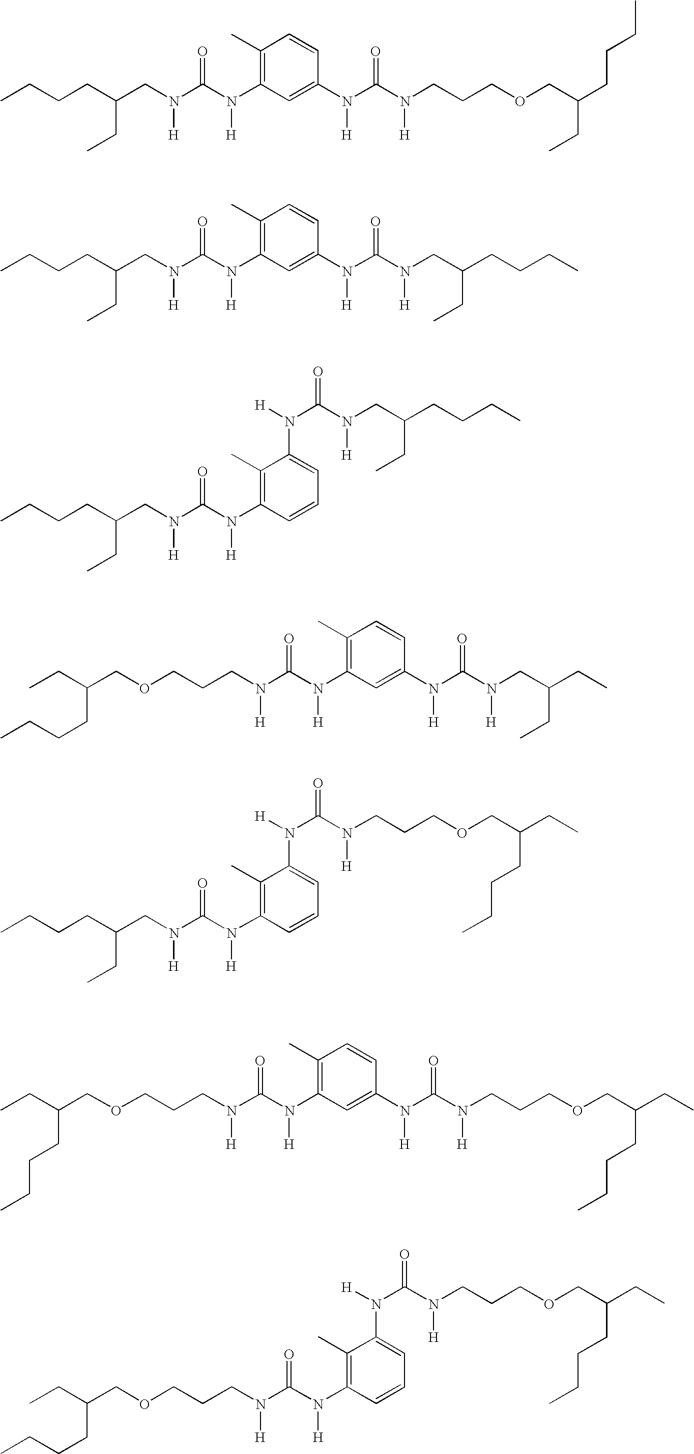

### EXAMPLE 4

The reaction mixture contains toluene duisocyanate as a mixture of the 2,4 isomer and the 2,6 isomer in a 95/5 proportion (m=0.52 g and n=3 mmol) and a mixture of amines consisting of 50 mol % of 2-ethyihexylamine (m=0.39 g and n=3 mmol) and 50 mol % of 3-(2-ethylhexyloxy)propylamine (m=0.56 g and n=3 mmol). The composition of the white powder obtained contains the same mixture of products as that obtained from Example 3, but in different proportions (confirmation by HPLC coupled to mass spectrometry).

### EXAMPLE 5

The reaction mixture contains toluene duisocyanate as a mixture of the 2,4 isomer and the 2,6 isomer in a 95/5 proportion (m=0.72 g and n=4.1 mmol) and a mixture of amines consisting of 0 mol % of tert-butylamine (m=0.31 g and n=4.2 mmol) and 50 mol % of 3-(2-ethylhexyloxy)propylamine (m=0.79 g and n=4.2 mmol). The composition of the white powder obtained contains the mixture of products below:

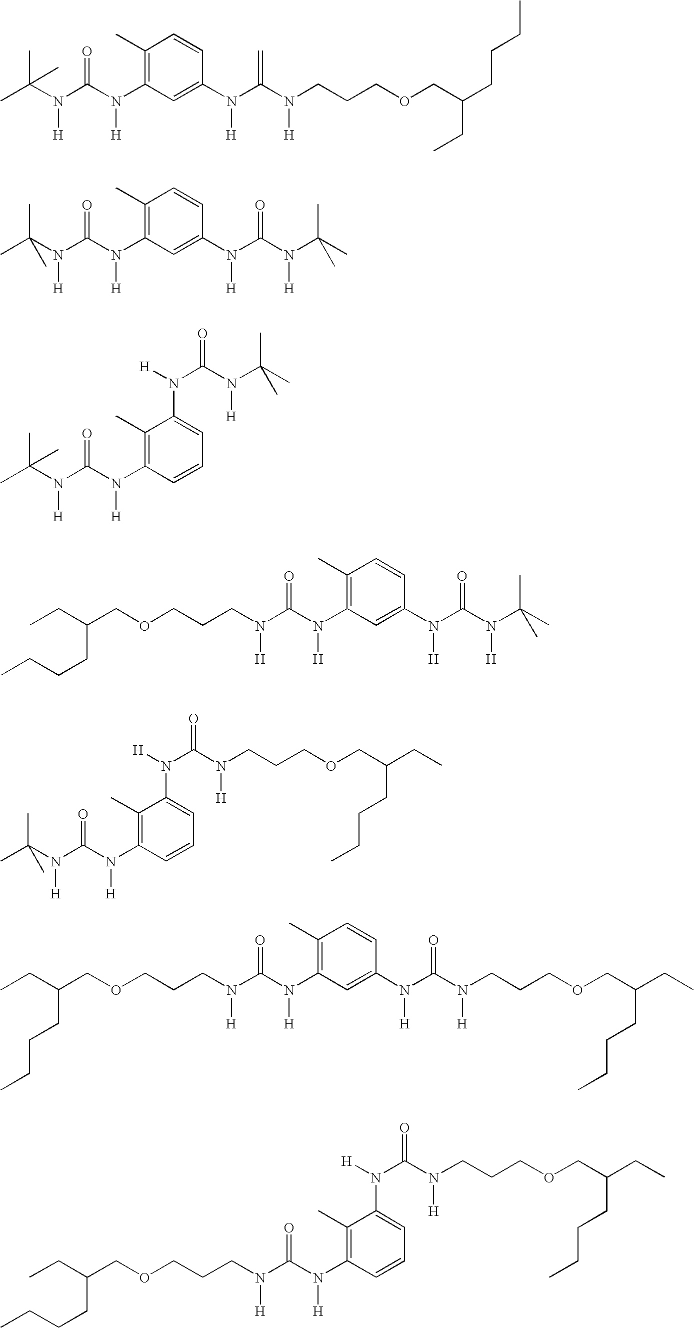

### EXAMPLE 6

The reaction mixture contains toluene diisocyanate as a mixture of the 2,4 isomer and the 2,6 isomer in a 95/5 proportion (m=2.44 g and n=14 mmol) and a mixture of amines consisting of 50 mol % of 2-ethylhexylamine (m=1.8 g and n=4 mmol) and 50 mol % of tert-butylamine (m=1.02 g and n=14 mmol). The composition of the white powder obtained contains the same mixture as that of Example 1, but in different proportions, confirmed by HPLC coupled to mass spectrometry.

### EXAMPLE 7

The reaction mixture contains toluene diisocyanate as a mixture of the 2,4 isomer and the 2,6 isomer in a 95/5 proportion (m=50 g and n=0.287 mol) and a mixture of amines consisting of 50 mol % of 2-ethylhexylamine (m=40.8 g and n=0.316 mol) and 50 mol % of ethyl 3-aminobutyrate (m=41.4 g and n=0.316 mol). The composition of the white powder obtained contains the mixture of products below:

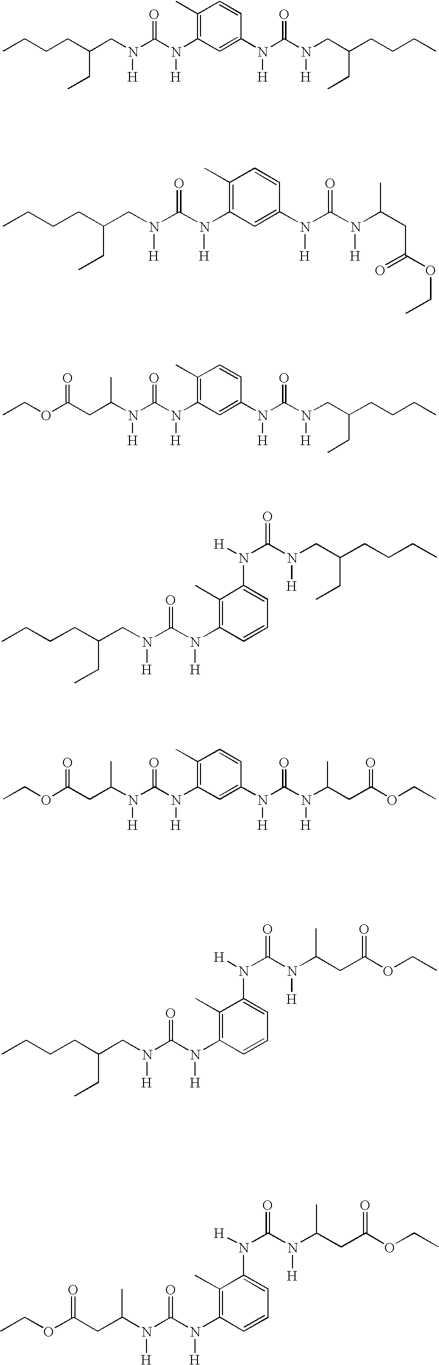

### EXAMPLE 8

### Example Starting with a Mixture of Three Amines

The reaction mixture contains toluene diisocyanate as a mixture of the 2,4 isomer and the 2,6 isomer in a 95/5 proportion (m=1.9 g; n=11 mmol) and a mixture of amines consisting of 80 mol % of 2-ethylhexylamine (m=2.2 g; n=17 mmol), of 10 mol % of 3-(2-ethylhexyloxy)propylamine (m=0.37 g; n=2 mmol) and 10 mol % of tert-butylamine (m=0.15 g; n=2 mmol).

### EXAMPLE 9

The symmetrical derivatives below were prepared for comparative purposes:

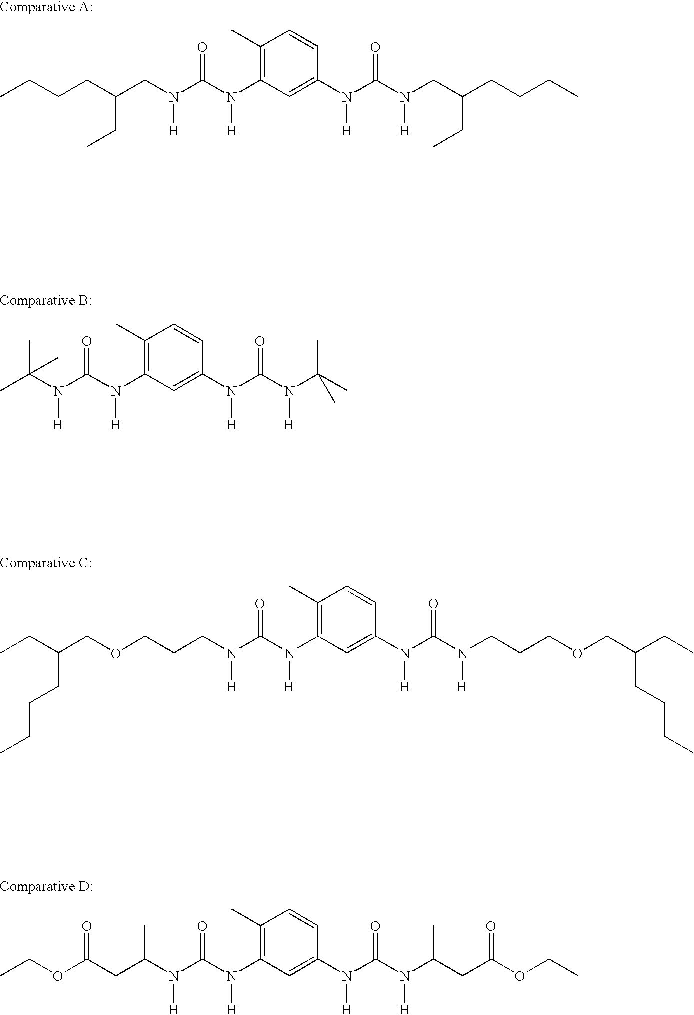

### EXAMPLE 10

The mixtures of compounds obtained according to Examples 1, 2, 3, 5 and 8 and the comparative compounds A, B, C and D of Example 9 are tested at a rate of 1% and 10% by weight for their gelling properties at room temperature with respect to a list of solvents.

To do this, an amount of the compound or mixture tested is introduced into an amount of solvent. The gelling properties are judged to be satisfactory if the compound or compound mixture dissolves at room temperature in the solvent and, where appropriate, if it increases the viscosity of this solvent.

It is noted that only the mixtures of Examples 1, 2, 3, 5 and 8 comprising a compound in accordance with the invention prove to be soluble at room temperature at concentrations of up to 10% by weight in phenyl trimethicones and increase the viscosity of these solvents.

On the other hand, the symmetrical derivatives of the Comparative Examples A, B and C do not prove to be soluble at 1% by weight in phenyl trimethicone, even at 80° C.

Similarly, it is observed that the mixtures of Examples 1, 2, 3, 5 and 8 are soluble at room temperature at 1% by weight in isododecane, in octyldodecanol, in isononyl isononanoate and in parleam oil, and bring about a substantial increase in the viscosity of these solvents.

On the other hand, the symmetrical derivatives of the Comparative Examples A, B and C do not prove to be soluble at 1% by weight in octyldodecanol, in isononyl isononanoate or in parleam oil, even at 80° C.

Furthermore, the mixtures of symmetrical derivatives of Examples A, B, C and D are not soluble and do not cause an increase in the viscosity of the medium.

By way of example: Example 1 is soluble at room temperature at 1% by weight in isododecane, in isononyl isononanoate and in parleam oil and causes an increase in the viscosity of the medium. On the other hand, this result is not observed by mixing 80% by mass of Comparative Example A with 20% by mass of Comparative Example B, (the mixture being in total at 1% by mass in the solvent), even on heating to 80° C.

The mixtures of Examples 6 and 7 are soluble at room temperature at 3% by weight in butyl acetate and cause, at this concentration, an increase in the viscosity of this ester oil. The same results are obtained with the mixture of Example 6 in ethyl acetate.

On the other hand, the symmetrical derivatives of the Comparative Examples A, B and D of Example 9 remain insoluble at 3% by weight in ethyl acetate and butyl acetate.

### EXAMPLE 11

Preparation of a Mascara

The composition is prepared using the following compounds:

| Name | Concentration mass % |
|---|---|
| Beeswax | 9.9 |
| Carnauba wax | 4.52 |
| Paraffin | 2.18 |
| Polyvinyl laurate¹ | 0.75 |
| Compound of Example 1 | 5 |
| Black iron oxide | 2.5 |
| Ultramarine blue | 2.1 |
| Preserving agent | 0.2 |
| Isododecane | qs |

¹Mexomer PP ® from CHIMEX

A mascara with entirely satisfactory cosmetic qualities is obtained.

1. A cosmetic composition with a liquid fatty phase, comprising, in a physiologically acceptable medium, at least one liquid fatty phase, in which said phase is textured with an effective amount of at least one compound of formula (I): 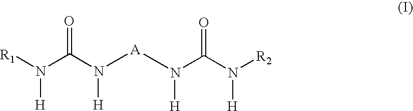
in which:
A is a group of formula: 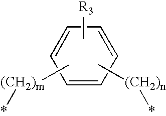
with
R₃ being a hydrogen atom or a linear or branched C₁ to C₄ alkyl radical,
n and m being, independently of each other, equal to 0 or 1, and
* symbolizing the point of attachment of the group A to the two nitrogen atoms of the residue of the compound of general formula (I),
R₁ is a saturated or unsaturated, non-cyclic branched C₃ to C₁₅ alkyl radical optionally containing from 1 to 3 heteroatoms selected from the group consisting of O, S, F and N and/or a carbonyl, and combinations thereof,
R₂ is different from R₁ and is chosen from linear, branched or cyclic, saturated or unsaturated C₁-C₂₄ alkyl radicals optionally containing from 1 to 3 heteroatoms selected from the group consisting of O, S, F and N, and optionally substituted with:
1, 2 or 3 hydroxyl radicals,
an ester radical (—COOR₄), with R₄ being a linear or branched alkyl radical containing from 1 to 8 carbon atoms;
a saturated, unsaturated or aromatic cyclic radical containing from 5 to 12 carbon atoms, optionally substituted with one or more radicals, which may be identical or different, selected from the group consisting of C₁-C₄ alkyl and trifluoromethyl radicals, or a morpholine derivative, and/or
one or more linear or branched C₁-C₄ alkyl radicals,
or a salt or isomer thereof.

2. The composition according to claim 1, in which the group A is a group of formula: 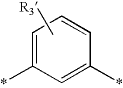
with R₃′ being a linear or branched C₁ to C₄ alkyl radical.

3. The composition according to claim 2, in which the group A is a group of formula: 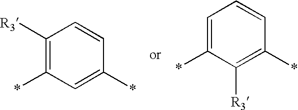
in pure form or as a mixture with the other positional isomers.

4. The composition according to claim 3, in which the group A is a group of formula:

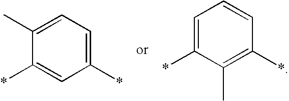

5. The composition according to claim 1, in which the group R₁ is selected from the group consisting of: 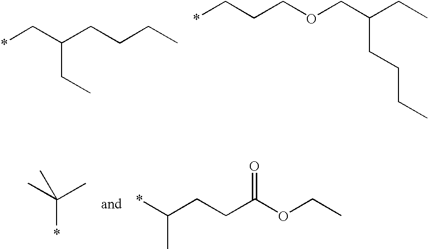
with * symbolizing the point of attachment of the group R₁ to the nitrogen of the residue of the compound of general formula (I).

6. The composition according to claim 1, in which the group R₂ is selected from the group consisting of: 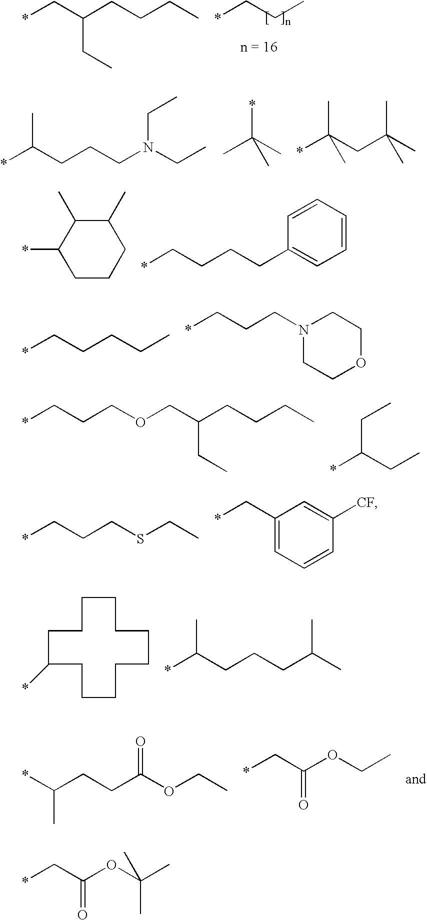
with * symbolizing the point of attachment of the group R₂ to the nitrogen of the residue of the compound of general formula (I).

7. The composition according to claim 1, in which the compound of formula (I) is selected from the group consisting of:

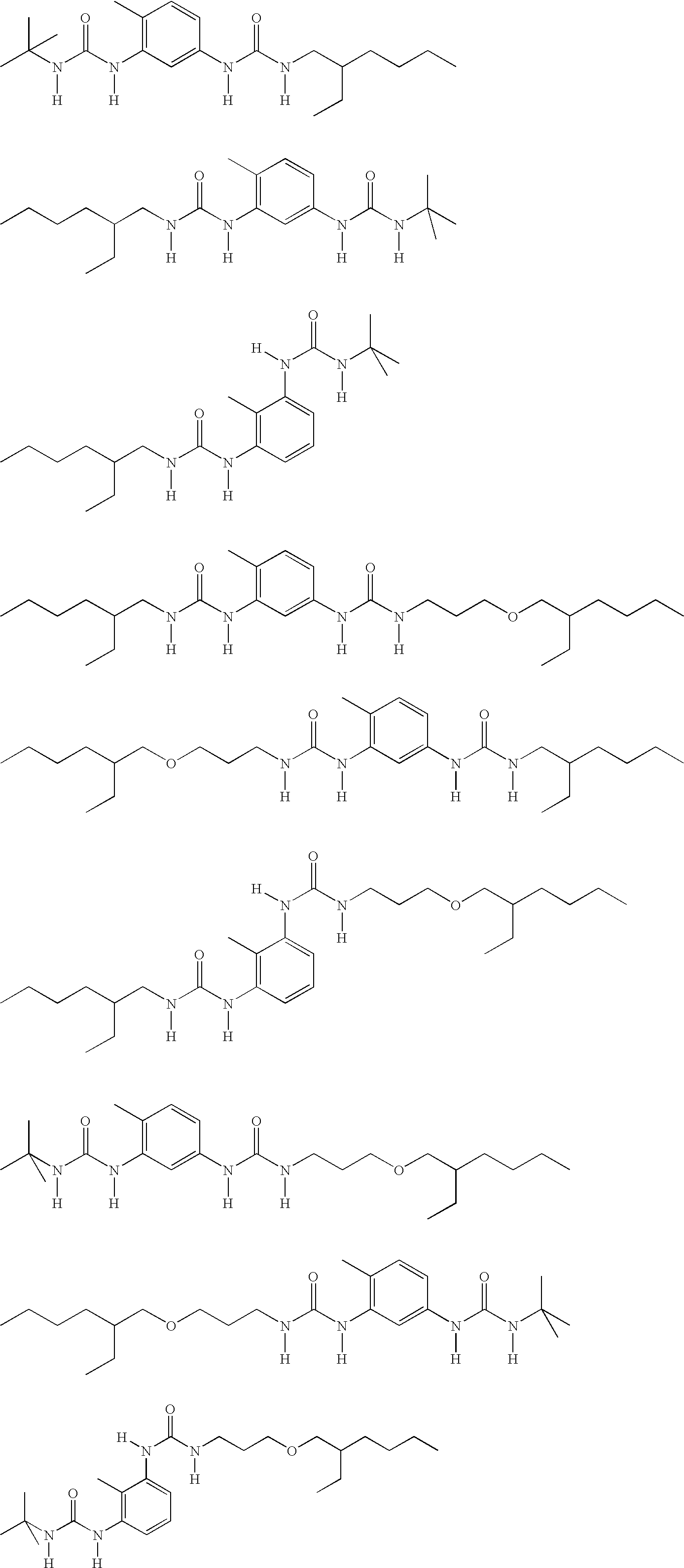

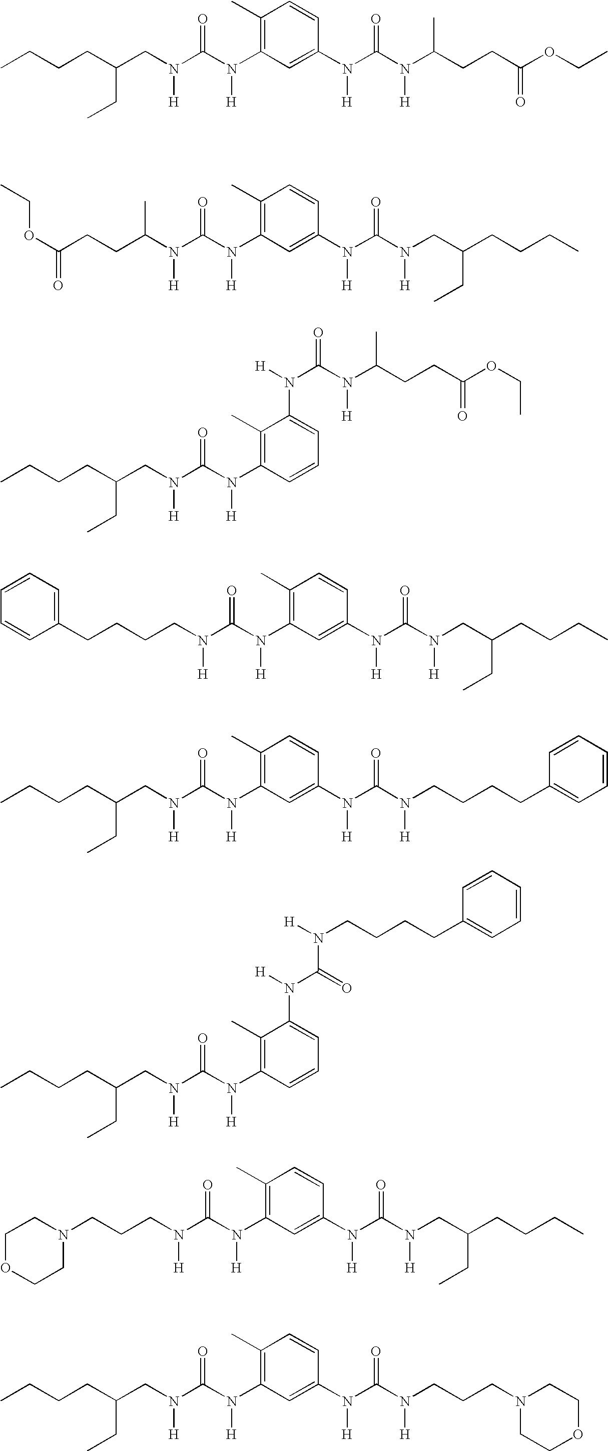

8. The composition according to claim 1, further comprising a gelling agent that comprises at least one compound of formula (II): 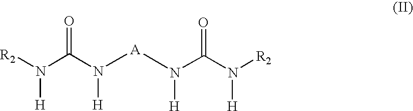
with A and R₂ as defined according to claim 1, A and R₂ having the same definition in formulae (I) and (II).

9. The composition according to claim 8, in which the gelling agent also comprises at least one compound of formula (D): 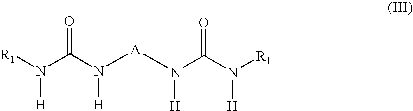
with A and R₁ as defined according to claim 1 and R₁ having the same definition in formulae (I) and (III).

10. The composition according to claim 1, in which the liquid fatty phase comprises at least one silicone oil selected from the group consisting of: polyalkylmethylsiloxanes; dimethicone copolyols, alkylmethicone copolyols; phenyl silicones, cetyl dimethicone, silicones containing alkylglyceryl ether groups, silicones containing amine side groups and dilauroyltrimethylolpropane siloxysilicate; fluorosilicones containing group(s) pendent or at the end of a chain, containing from 1 to 12 carbon atoms, all or some of the hydrogen atoms of which are replaced with fluorine atoms, and mixtures thereof.

11. The composition according to claim 1, in which the liquid fatty phase comprises at least one ester oil selected from the group consisting of the esters of formula (IV):
R₁—CO—O—R₂ (IV)
in which:
R₁ represents a linear or branched alkyl radical of 1 to 40 carbon atoms optionally comprising one or more ethylenic double bonds, and optionally substituted, and
R₂ represents a linear or branched alkyl radical of 1 to 40 carbon atoms, optionally comprising one or more ethylenic double bonds, and optionally substituted.

12. The composition according to claim 1, in which the liquid fatty phase comprises at least one hydrocarbon-based oil or one volatile ether selected from the group consisting of hydrocarbon-based plant oils, C₆-C₄₀ synthetic ethers, C₈-C₃₂ fatty alcohols and C₈-C₃₂ fatty acids, and mixtures thereof, liquid paraffins and hydrogenated polyisobutenes, and/or mixtures thereof

13. The composition according to claim 1, in which the liquid fatty phase comprises at least one oil selected from the group consisting of: octyldodecanol, Parleam, isododecane, isononyl isononanoate, a phenyl trimethicone, isopropyl N-lauroylsarcosinate (Eldew 205), ethyl acetate and butyl acetate, and mixtures thereof.

14. The composition according to claim 1, in which said compound of formula (I) is derived from a reaction between at least one diisocyanate of formula: 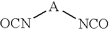
and at least two different primary amines of formulae:

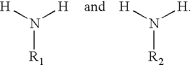

15. The composition according to claim 14, in which the number of primary amines used is two and the mole ratio n(R₁)/n(R₂) ranges between 10/90 and 90/10,
with n(R₁) corresponding to the number of moles of: 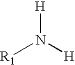
and n(R₂) corresponding to the number of moles of:

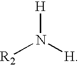

16. The composition according to claim 14, in which the number of amines used for the reaction is greater than or equal to 2.

17. The composition according to claim 14, in which the amines used are, as a whole, in a mole ratio of 2 to 3 equivalents per one equivalent of diisocyanate.

18. The composition according to claim 1, comprising less than 0.5% by weight of compound(s) of general formula (I) relative to the total weight of the liquid fatty phase.

19. The composition according to claim 1, also comprising at least one dyestuff.

20. The composition according to claim 1, also comprising at least one filler.

21. The composition according to claim 1, also comprising at least one cosmetic or dermatological active agent.

22. The composition according to claim 1, also containing at least one additive selected from the group consisting of water, vitamins, thickeners other than those corresponding to the general formula (I), gelling agents other than those corresponding to the general formula (I), trace elements, softeners, sequestrants, fragrances, acidifying or basifying agents, preserving agents, sunscreens, surfactants, antioxidants, hair-loss counteractants, antidandruff agents, propellants and ceramides, and mixtures thereof.

23. (canceled)

24. Process for caring for and/or making up a keratin material comprising the application to the surface to be treated of a composition according to claim 1.
